(19) United States
(12) Patent Application Publication
(10) Pub. No.: US 2012/0283526 A1
(43) Pub. Date: Nov. 8, 2012

(54) Method and a System for the Prediction of Epileptic Seizures

(75) Inventors: Kim Gomme Gommesen, Odense (DK); Karsten Hoppe, Copenhagen (DK)

(73) Assignee: ICTALCARE A/S, Hørsholm (DK)

(21) Appl. No.: 13/516,802

(22) PCT Filed: Dec. 15, 2010
(86) PCT No.: PCT/DK2010/000176

§ 371 Date: Jul. 26, 2012

(30) Foreign Application Priority Data
Dec. 16, 2009 (DK) PA 2009 01330

(51) Int. Cl. A61B 5/0205; A61B 5/0402; A61B 5/024; A61B 5/00

(52) U.S. Cl. 600/301; 600/300; 600/509; 600/508; 600/483

(57) ABSTRACT

The invention relates to a system and a method for the prediction of epileptic seizures by continuous measurement of at least one signal on the body of a user. A sensor unit measures the heart rate continuously and is connected to a processor unit. The processor unit comprises a verification unit, which stores those measurements that comprise verifiable heartbeats or heart rates in a database. An analysis unit in the processor analyzes the verified measurements to determine whether a preictal phase is present or not. The processor comprises a summation unit, which continuously determines the value of an indication signal on the basis of the signal from the analysis unit. The signal indicates the probability of an imminent epileptic seizure. The value of the indication signal may be determined on the basis of at least two values of the signal from the analysis unit. An alarm unit connected to the processor unit generates one or more alarms, if the indication signal exceeds one or more alarm levels.

This application claims the benefit of Danish Application No. PA 2009 01330 filed Dec. 16, 2009 and PCT/DK2010/000176 filed Dec. 15, 2010, International Publication No. WO 2011/072684, which are hereby incorporated by reference in their entirety as if fully set forth herein.

### TECHNICAL FIELD

The present invention relates to a system for the prediction of epileptic seizures and comprising a sensor unit intended to record a physiologically, neurologically or muscularly created signal on the body of a user, a processor unit connected to the sensor unit and intended to compare the sensor signal with reference parameters and to generate an indication signal indicating whether a preictal phase is present or not, and an alarm unit connected to the processor unit and intended to generate an alarm on the basis of the indication signal.

The invention also relates to a method of predicting epileptic seizures and comprising the following steps: recording a physiologically, neurologically or muscularly created signal on the body of a user by means of a sensor unit, comparing the sensor signal with reference parameters in a processor unit, and generating an indication signal when an alarm state has been recorded, and generating an alarm signal in an alarm unit.

### THE PRIOR ART

Various attempts at predicting epileptic seizures are described in the literature. An example of an apparatus and a method is described in WO 99/56821 A1, which describes an implantable electrode and sensor implanted in the body of a patient, preferably in the head, connected to an implantable processor and signal generator which is capable of emitting a stimulating signal via an implantable electrode. The processor is capable of recognizing various patterns and of applying a signal to the signal generator to generate a stimulating signal, so that the person is warned of a seizure.

WO 2007/072425 A2 describes a cuff/band containing a plurality of sensors connected to a processor, which is capable of recognizing various patterns characteristic of an epileptic seizure and of applying an alarm signal to an alarm unit which generates an alarm.

US 2008/0161713 A1 describes a system for the prediction of epileptic seizures and comprising a plurality of electrodes connected to a communications unit, which is capable of analyzing the data measured, and which communicates with an external data unit which is capable of giving an alarm or instructions to the user. The communications unit comprises several processor units which extract parameters from the signal measured. The parameters are used by several classification units for classifying the state measured. The classification units are capable of giving a weighted answer which can indicate the probability of an imminent seizure within a predetermined time frame. The system determines the time interval of the coming measurements on the basis of the value of the weighted answer.

Attempts at predicting seizures in newborns based on ECG measurements of the heart rate are described in the literature (MALARVILI & MESBAH, “Newborn seizure detection based on heart rate variability”). In the attempt, the entire measurement is stored, following which the heart rate parameters are extracted by means of a QRS algorithm. Then, the heart rate parameters are analyzed both in the time domain and in the time frequency domain to select the suitable data by means of a selection process. The data are then classified in a plurality of classes, which define seizure and non-seizure.

The known systems, however, have the drawback that either they must be implanted in the body, or their structure makes them difficult to position on the body. A drawback of the known systems is also that they only carry out measurements periodically and measure and store a multitude of superfluous measurements.

However, there is no system or method which has a structure that is easy to attach to the body, and which makes continuous measurements on the body, where only verifiable measurements are stored and processed by the processor.

### THE OBJECT OF THE INVENTION

The present invention remedies the problems of the most immediate prior art by providing a system for the prediction of epileptic seizures, characterized in that the sensor unit performs continuous measurements, and that the sensor unit is connected to a verification unit in the processor unit, which compares the sensed signal with predetermined parameters characteristic of the measured signal and stores only verifiable measurements, and that the processor unit compares the present measurement with one or more preceding measurements to determine the value of the indication signal, which is transmitted to the alarm unit. This ensures a reduction in the data amount of recorded measurements, while improving the possibility of recording a preictal phase, since the subsequent evaluation is only performed on the basis of verified measurements.

According to one embodiment of the invention, the processor unit comprises an analysis unit, which compares the verified measurement with the reference parameters descriptive of the signal measured under normal conditions, and which generates a first indication signal indicating whether a preictal phase is present or not. Hereby, it is possible to detect a preictal phase more precisely on the basis of one or more predetermined criteria.

According to a specific embodiment of the invention, the processor unit comprises a summation unit, which compares the present value of the first indication signal with the previous value or at least two of the preceding values of the first indication signal to determine the value of a second indication signal, which is transmitted further on to the alarm unit. According to a further specific embodiment of the invention, the value of the second indication signal is increased by a predetermined value, either if the present value and the previous value or at least two of the preceding values of the first indication signal are high, of if the number of high values is greater than the number of low values. According to a further specific embodiment of the invention, the value of the second indication signal is reduced by a predetermined value, either if the present value and the previous value or at least two of the preceding values of the first indication signal are low, or if the number of low values is greater than the number of high values. According to a further specific embodiment of the invention, the value of the second indication signal remains unchanged, either if the present value and the previous value of the first indication signal are different, or if the number of low values is equal to the number of high values. This results in a continuous evaluation of the probability of an imminent epileptic seizure. Moreover, it is possible to average over several time periods, thereby compensating for fast variations in the preictal signal.

According to one embodiment of the invention, the values of the first indication signal are summed, and the sum is compared with one or more threshold values indicating whether the value of the second indication signal is increased, is reduced or remains unchanged.

According to one embodiment of the invention, the processor unit is connected to a database, and the reference parameters are stored in the database and describe the characteristic of the measured signal under normal conditions. According to a specific embodiment of the invention, a self-learning process is implemented in the processor unit, which automatically updates the reference parameters stored in the database and optionally adds new reference parameters to the database. This results in a more detailed characteristic of the signal, and the parameters of the characteristic may be adjusted as the characteristic changes.

According to one embodiment of the invention, the sensor unit comprises a heart rate sensor intended to measure an electrocardiographic signal of the heart rate or to detect another signal representative of the heart rate, such as pulse, blood pressure or a photoplethysmographic signal.

According to one embodiment of the invention, the sensor unit comprises at least an electrode or a sensor connected via a cable or wirelessly connected either directly to the processor or to a local unit, which is in turn connected to the processor via a cable or a wireless connection. This results in an optimum measurement of one or more signals in the body.

According to one embodiment of the invention, the sensors or electrodes of the sensor unit and associated electronics are incorporated in the same unit, so that the heart rate is measured at a point. This makes it possible to measure the signal at a single point, thereby ensuring a simple and easy way of attaching the unit firmly to the body.

According to a specific embodiment of the invention, the processor unit is connected to at least a second sensor in the sensor unit or at least a second sensor unit, such as an electroencephalographic sensor, an electromyographic sensor, an electrocardiographic sensor, a gyrometer, or an accelerometer, intended to measure at least another physiologically, neurologically or muscularly crated signal, such as breathing, temperature, perspiration, muscular tensions, tremors/convulsions or a galvanic skin response. According to a further specific embodiment of the invention, the second sensor or sensor unit is connected to the analysis unit optionally via at least a second verification unit. This results in a more precise recording of a preictal phase.

The present invention remedies the problems of the most immediate prior art additionally by providing a method of predicting epileptic seizures, characterized in that the signal is measured continuously and compared with predetermined parameters characteristic of the measured signal, and only verified measurements are stored and processed in the processor unit, and that the processor unit continuously compares the present measurement with one or more preceding measurements to change the value of the indication signal, which is transmitted to the alarm unit. This results in a reduction in the data amount of recorded measurements, while improving the possibility of recording a preictal phase, since the subsequent measurement is only performed on the basis of verified measurements.

According to one embodiment of the invention, the processor unit compares the verified measurement with the reference parameters and generates a first indication signal indicating whether a preictal phase is present or not. This makes it possible to detect a preictal phase more precisely on the basis of one or more predetermined criteria.

According to a specific embodiment of the invention, the processor unit determines the value of a second indication signal, which is transmitted further on to the alarm unit, on the basis of the present value of the first indication signal and the previous value or at least two of the preceding values of the first indication signal. According to a further specific embodiment of the invention, the value of the second indication signal is increased by a predetermined value, either if the present value and the previous value or at least two of the preceding values of the first indication signal are high, or if the number of high values is greater than the number of low values. According to a further specific embodiment of the invention, the value of the second indication signal is reduced by a predetermined value, either if the present value and the previous value or at least two of the preceding values of the first indication signal are low, or if the number of low values is greater than the number of high values. According to a further specific embodiment of the invention, the value of the second indication signal remains unchanged, either if the present value and the previous value of the first indication signal are different, or if the number of low values is equal to the number of high values. This results in a continuous evaluation of the probability of an imminent epileptic seizure. Moreover, it is possible to average over several time periods, thereby compensating for fast variations in the preictal signal.

According to one embodiment of the invention, the values of the first indication signal are summed, and the sum is compared with one or more threshold values indicating whether the value of the second indication signal is increased, is reduced or remains unchanged.

According to one embodiment of the invention, the signal is measured under normal conditions and is stored in a database as reference signals, and the reference parameters are updated automatically, and possibly new reference parameters are added to the database by means of a self-learning process implemented in the processor unit. This ensures a more detailed characteristic of the signal, and the parameters of the characteristic may be adjusted as the characteristic changes.

According to one embodiment of the invention, a heart rate sensor measures an electrocardiographic signal of the heart rate or detects another signal representative of the heart rate, such as pulse, blood pressure or a photoplethysmographic signal, or a first sensor measures another physiologically, neurologically or muscularly created signal than the heart rate, such as breathing, temperature, perspiration, muscular tensions, tremors/convulsions or galvanic skin response.

According to one embodiment of the invention, the heart rate is measured at a point by means of an electrode or a sensor in the sensor unit, which transmits data further on to the processor either via a cable or wirelessly. This makes it possible to measure the signal at a single point, which ensures a simple and easy way of attaching the unit firmly to the body.

According to one embodiment of the invention, at least a second sensor in the sensor unit or at least a second sensor unit measures at least another physiologically, neurologically or muscularly created signal than the heart rate, such as breathing, temperature, perspiration, muscular tensions, tremors/convulsions or galvanic skin response. According to a specific embodiment of the invention, the measurement from the second sensor or sensor unit is compared with the measurement of the first signal in the analysis unit. This ensures a more precise recording of a preictal phase.

### THE DRAWING

Exemplary embodiments of the invention will be explained more fully below with reference to the drawing, in which

FIG. 1 shows a basic sketch of the invention, and

FIG. 2 shows a block diagram of how the invention operates.

### DESCRIPTION OF EXEMPLARY EMBODIMENTS

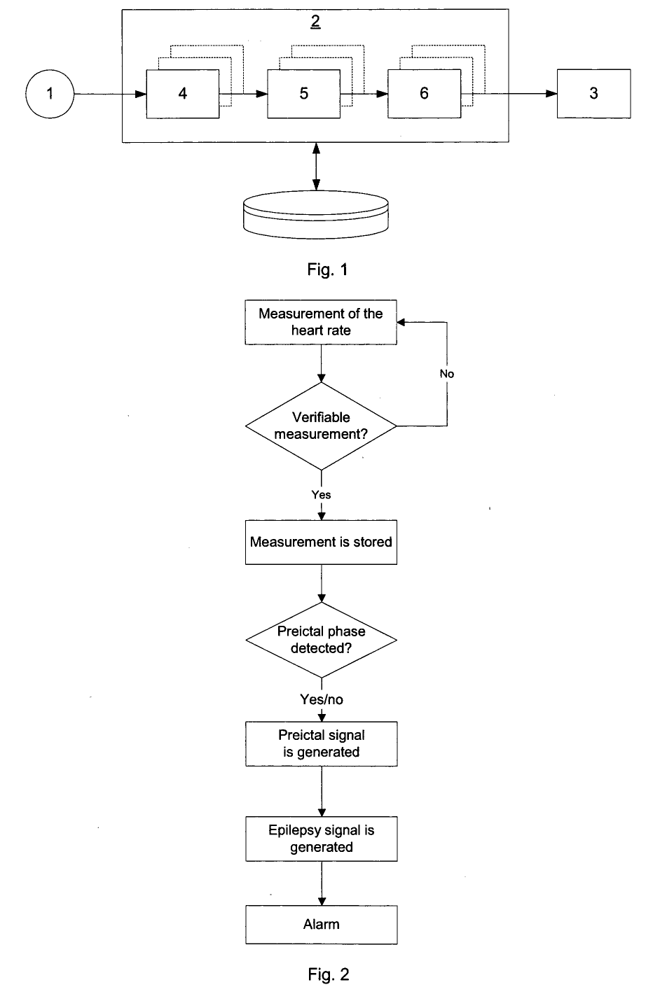

With reference to FIG. 1, this figure shows a basic sketch of the invention which contains a sensor unit 1, a processor unit 2 and an alarm unit 3.

The sensor unit 1 is placed on the body of a user and records one or more signals generated in the body. The sensor unit 1 may contain at least a primary sensor and/or electrode, which is intended to measure a specific signal and is placed strategically on the body relative to the measurement of the signal concerned.

The processor unit 2 is connected to the sensor unit 1 and stores the signals which may be verified according to the concrete measurement. The verified signals are then analyzed to decide whether a state characteristic of a preictal phase is present or not. If the state is present, an alarm signal is generated and transmitted to the alarm unit 3.

The alarm unit 3 is connected to the processor unit 2 and generates an alarm on the basis of the alarm signal, so that the user is warned of an imminent seizure.

The sensor unit 1 may comprise one or more electrodes placed at various places on the body and may be connected to the processor unit 2. Alternatively, the sensor unit 1 may comprise one or more sensors placed at various places on the body, all of which may be connected directly to the processor unit 2 or to a local unit on the body, which is capable of transmitting data further on to the processor unit 2 via a wireless connection or a cable. The sensor unit 1 may also be placed in the clothing that surrounds a portion of the body, or be combined with other types of sensors placed on the body, which measure the same signal or other signals. When the electrode or sensor of the sensor unit 1 is placed strategically, an optimum measurement of one or more signals in the body may be achieved.

In a preferred embodiment, the sensors and/or electrodes of the sensor unit 1 and associated electronics are incorporated in one and the same unit. When the signal is measured at a single point, a simple and easy way of attaching the unit firmly to the body may be achieved.

The sensor and/or the electrode may be arranged in a device which may be secured to a specific point on the body. The device may comprise an adhesive layer arranged on the lower side of a flexible or bendable material, such as rubber, plastics or another material of the same properties which is capable of conforming to the contours of the body. Alternatively, the device may be configured as a plaster or an electronic plaster, which comprises a sensor system embedded or encapsulated in an adhesive device. Alternatively, a gel or paste may be used for securing the device to the body. This makes it possible to secure the sensor unit to the body and to bring the sensor or the electrode into contact with the body.

The signals measured are any physiologically, neurologically or muscularly created signal which is characteristic of an epileptic seizure, including any electroencephalographic, electromyographic or electrocardiographic signal which is characteristic of any epileptic seizure.

In a preferred embodiment, the electrocardiographic signal of the heart is measured by any type of heart rate sensor, including also electromyographic sensors intended to measure the electrocardiographic signal. A heart rate sensor may be disposed above the heart or at another place on the body, or at least two heart rate sensors may be disposed strategically on the chest. Alternatively, the heart rate may also be measured by means of another type of heart rate sensor, which measures the heart rate by detecting another signal than the electrocardiographic signal, e.g. by measuring the pulse, the blood pressure or a photoplethysmographic (PPG) signal.

To achieve a more precise recording of a preictal phase which occurs just before an epileptic seizure, measurement of the heart rate may be combined with other measurements, such as breathing, temperature, perspiration, muscular tensions, tremors/convulsions or neural response. The measurement of at least a second signal may be carried out by means of at least a second sensor in the sensor unit 1, or at least a second sensor unit which may be connected to the processor unit 2. The second sensor or sensor unit may be an electroencephalographic sensor, an electromyographic sensor, an electrocardiographic sensor, a gyrometer, an accelerometer or another type of sensor intended to perform the desired measurement.

Alternatively, the heart rate measurement may be combined with the measurement of galvanic skin response of the body performed at at least one strategically selected place on the body. The measurement of the galvanic skin response may be performed by at least a second sensor, or at least a second sensor system which may be connected to the processor unit 2.

In a first preferred embodiment, the sensor unit 1 performs a continuous measurement of the heart rate and heartbeats, which are transmitted to the processor unit 2 either wirelessly or via a cable.

The processor unit 2 comprises a verification unit 4 connected to the sensor unit 1.

The verification unit 4 may comprise an amplifier part, in which the measurements from each individual sensor or electrode are amplified to a suitable signal level. Alternatively, the amplifier part may be integrated into the sensor unit 1.

Then, the recorded measurements may be filtered or compared with a plurality of predetermined parameters characteristic of a heartbeat or a heart rate. The parameters may be amplitude values, time intervals, frequencies or corresponding parameters which are characteristic of a heartbeat. The parameters may be stored in a memory or database, in which their values may be updated currently, and new criteria may be added. Alternatively, also sequences (patterns) characteristic of various heart rates may be stored in the memory or the database.

If the measurement performed over a given time period contains a verifiable heartbeat or heart rate, the measurement is stored in the memory, if not, the measurement is disregarded, as shown in FIG. 2. Hereby, the data amount of recorded measurements is reduced, and the possibility of recording a preictal phase is improved, since the subsequent evaluation is only performed on the basis of verified measurements.

Alternatively, the processor unit 2 may also contain at least a second verification unit which is intended to verify measurements of at least another physiologically, neurologically or muscularly created signal measured on the body, and which may be connected to the sensor unit 1 and/or at least a second sensor unit.

The processor unit 2 comprises a preictal analysis unit 5, which continuously compares the verified measurements with a plurality of different reference parameters stored in the memory or the database, as shown in FIG. 2. The reference parameters describe the heart rate and the heartbeats in a plurality of different non-seizure related states, which, in combination, give a detailed characteristic of the heart under normal conditions.

The reference parameters in the database or the memory may be updated currently, or new parameters may be added using a learning process controlled by the processor unit 2. Hereby, it is possible to achieve a more detailed characteristic of the heart and also to adjust the parameters of the characteristic, as the characteristic of the heart changes.

In the preferred embodiment, the reference parameters are determined by performing a plurality of measurements over a plurality of given time periods, so that the heart rate and the heartbeats are measured under the various activities which may occur during one or more normal days of the user. The activities may be normal movement of the musculature, sport exercises, sleep, psychic influences, impacts during work and other natural/normal activities in normal everyday life. The sensor unit 1 measures the strain on the heart continuously during these activities, where the heartbeats and the heart rate are verified and stored in the memory in the processor unit 2. Hereby, it is possible to determine a detailed characteristic of the heart during normal conditions.

The preictal analysis unit 5 is capable of comparing the parameters of the measured heartbeats with the reference parameters saved in the memory or the database. Also, the parameters of the measured heart rate may be compared with the reference parameters saved in the memory or the database. Alternatively, also measured heart rate sequences (patterns) may be compared with the reference parameters saved in the memory or the database. Hereby, it is possible to detect a preictal phase more precisely on the basis of one or more predetermined criteria.

Alternatively, the heart rate measurement may be compared with the measurement of at least another physiologically, neurologically or muscularly created signal, so that the criteria which are characteristic of a preictal phase, may be determined more precisely. The criteria may be increasing loss of consciousness, increasing temperature, increased perspiration, involuntary motor movements and increased shallow breathing. Hereby, it is possible to achieve a more precise recording of a preictal phase on the basis of several different criteria.

In an alternative embodiment, the preictal analysis unit 5 may be implemented as a cluster analysis algorithm, which is capable of dividing the stored reference measurements into a plurality of clusters or groups in accordance with at least one criterion. The clusters or the groups describe the characteristic of the heart under normal conditions of the user. Then, the algorithm may compare the heart rate measurement with the clusters or the groups to determine whether a preictal phase is present or not.

If a preictal phase is present, the preictal analysis unit 5 generates a high preictal signal. If a preictal phase is not present, the preictal analysis unit 5 generates a low preictal signal.

Alternatively, the analysis unit 5 may generate a weighted preictal signal, which is determined on the basis of which one or ones of the criteria characteristic of a preictal phase has/have been detected. Alternatively, the analysis unit 5 may weight the various verified measurements performed by the sensor units 1 relative to each other, before the analysis unit generates the preictal signal.

The processor unit 2 comprises a summation unit 6, which continuously changes the value of an epilepsy signal indicating the probability of an imminent epileptic seizure, as shown in FIG. 2. On the basis of the preictal signal, the summation unit 6 calculates how long a preictal phase has been present over a given time period. The predetermined time period may be equal to or different from the time period or periods over which the measurements are performed. If the present preictal signal has a high value, the value of the epilepsy signal is increased by a predetermined value. If the preictal signal has a low value, the epilepsy signal is reduced by the same value. If the epilepsy signal has a high value, it indicates a great probability of an imminent epileptic seizure, while a low value indicates a small probability of an imminent epileptic seizure. The calculations may take place by simple summation and subtraction within a given interval, such as from 0 to 100, or by another mathematic operation. Hereby, it is possible to evaluate the probability of an imminent epileptic seizure continuously.

Alternatively, the summation unit 6 may compare the present preictal signal with one or more of the preceding preictal signals. If the value of the present preictal signal is high, and the values of the previous preictal signal or at least two of the preceding preictal signals are high, the value of the epilepsy signal is increased by the predetermined value. If the value of the present preictal signal is low, and the values of the previous preictal signal or at least two of the preceding preictal signals are low, the value of the epilepsy signal is decreased by the predetermined value. If the value of the present preictal signal and the value of the previous preictal signal are different, or if the number of high preictal signals and the number of low preictal signals are the same, the value of the epilepsy signal remains unchanged. Hereby, it is possible to average over several time periods, thereby compensating for fast variations in the preictal signal.

In a further alternative embodiment, the summation unit 6 may sum the value of the present preictal signal with the value of at least one or more of the previous preictal signals. The summed value may then be compared with one or more threshold values, where each level indicates whether the value of the epilepsy signal is to be increased or reduced by a predetermined value or remain unchanged.

The memories or the databases connected to the processor unit 2 may be implemented as individual memory areas in a single memory. Alternatively, the processor unit 2 may be connected to at least two separate memories, which are connected to their respective units in the processor.

The epilepsy signal is transmitted to an alarm unit 3, which is capable of warning the user of an imminent seizure, as shown in FIG. 2. The alarm unit 3 may comprise one or more comparators, which compare the epilepsy signal with one or more alarm levels capable of activating their separate alarm circuits. The comparators may be connected to one or more alarm circuits, which are capable of generating a visual alarm, an acoustic alarm, vibrations or another form of alarm indication. Alternatively, the alarm circuit is capable of generating two or more alarms of the same type or a combination of various alarm types. Alternatively, the alarm unit 3 may transmit an external alarm signal to an external system, which is capable of performing a suitable act on the basis of the alarm signal. Hereby, it is possible to warn the user of an imminent seizure via one or more alarm signals, so that the user has the possibility of acting before the seizure.

The learning process is implemented in the processor unit 2 and is controlled by a controller in the processor unit 2. The controller controls each unit 4, 5, 6 in the processor and may alternatively be connected to an I/O unit, which may be connected to a user interface, so that the controller may receive external instructions. Hereby, it is possible that one or more processes, including the learning process, may be activated externally.

In the preferred embodiment, the learning process may be implemented as an automatic, self-learning process capable of recording and storing new heart rate measurements or updating existing heart rate measurements. The process may verify and compare the measurement with the reference measurements. If the measurement has already been stored in the memory or the database, the parameters are updated. If the measurement has not been stored in the memory or the database, the parameters for the new measurement are stored. Hereby, it is possible to update the characteristic of the heart automatically, thereby improving the possibility of detecting an epileptic seizure.

In another preferred embodiment, the same system as described above may be used for predicting epileptic seizures on the basis of another physiologically, neurologically or muscularly created signal measured on the body of the user. In this embodiment, the heart rate sensor is replaced by another type of sensor intended to measure the signal concerned, e.g. breathing, temperature, perspiration, muscular tensions, tremors/convulsions or galvanic skin response. The sensor may be an electroencephalographic sensor, an electromyographic sensor, an electrocardiographic sensor, a gyrometer, an accelerometer or another type of sensor intended to perform the desired measurement.

Moreover, the reference parameters stored in the memory or the database do not describe the characteristic of the heart, but the characteristic of the measured signal under normal conditions, and are determined in the same manner as described in the first preferred embodiment.

The processor units 4, 5, 6 and the alarm unit 3 have the same structure as described in the first preferred embodiment. The verification units 4 and the analysis units 5 are adapted to the measurement of the signal concerned.

As described in the first preferred embodiment, the measured signal may be compared with other measured physiologically, neurologically or muscularly created signals to determine a preictal phase more precisely.

The invention is not limited to the structure indicated in the preferred embodiment. The preferred embodiment may be combined with any structure indicated in the alternative embodiments.

1. A system for the prediction of epileptic seizures, comprising
a sensor unit (1) intended to record a physiologically, neurologically or muscularly created signal on the body of a user,
a processor unit (2) connected to the sensor unit and intended to compare the sensor signal with reference parameters and to generate an indication signal indicating whether a preictal phase is present or not, and
an alarm unit (3) connected to the processor unit and intended to generate an alarm, wherein
the sensor unit (1) performs continuous measurements, and that the sensor unit is connected to a verification unit (4) in the processor unit, which compares the sensed signal with predetermined parameters characteristic of the measured signal and stores only verifiable measurements, [p. 8, 1. 21-25, p. 11, 1. 8-19] and
that the processor unit (2) continuously compares the present measurement with one or more preceding measurements to determine the value of the indication signal, which is transmitted to the alarm unit (3).

2. A system according to claim 1, wherein the processor unit comprises an analysis unit (5), which compares the verified measurement with the reference parameters descriptive of the measured signal under normal conditions, and which generates a first indication signal indicating whether a preictal phase is present or not [p. 11, 1. 30-p. 12, 1.4].

3. A system according to claim 2, wherein the processor unit comprises a summation unit (2), which compares the present value of the first indication signal with the previous value or at least two of the preceding values of the first indication signal to determine the value of a second indication signal, which is transmitted further on to the alarm unit (3).

4. A system according to claim 3, wherein the value of the second indication signal is increased by a predetermined value, either if the present value and the previous value or at least two of the preceding values of the first indication signal are high, or if the number of high values is greater than the number of low values.

5. A system according to claim 3, wherein the value of the second indication signal is reduced by a predetermined value, either if the present value and the previous value or at least two of the preceding values of the first indication signal are low, or if the number of low values is greater than the number of high values.

6. A system according to claim 3, wherein the value of the second indication signal remains unchanged, either if the present value and the previous value of the first indication signal are different, of if the number of low values is equal to the number of high values.

7. A system according to claim 3, wherein the values of the first indication signal is summed, and the sum is compared with one or more threshold values indicating whether the value of the second indication signal is increased, is reduced or remains unchanged.

8. A system according to claim 1, wherein the processor unit (2) is connected to a database, and that the reference parameters are stored in the database and describe the characteristic of the measured signal under normal conditions [p. 12, 1. 2-10].

9. A system according to claim 8, wherein a selflearning process is implemented in the processor unit (2), which automatically updates the reference parameters stored in the database and optionally adds new reference parameters to the database.

10. A system according to claim 1, wherein the sensor unit (1) comprises
a heart rate sensor intended to measure an electrocardiographic signal of the heart rate or to detect another signal representative of the heart rate, such as pulse, blood pressure or a photoplethysmographic signal, or
a first sensor, such as an electroencephalographic sensor, an electromyographic sensor, an electrocardiographic sensor, a gyrometer or an accelerometer, intended to measure another physiologically, neurologically or muscularly created signal than the heart rate, such as breathing, temperature, perspiration, muscular tensions, tremors/convulsions or galvanic skin response.

11. A system according to claim 10, wherein the sensor unit (1) comprises at least an electrode or a sensor connected via a cable or wirelessly connected either directly to the processor (2) or to a local unit, which is in turn connected to the processor (2) via a cable or a wireless connection.

12. A system according to claim 10, wherein the sensors or electrodes of the sensor unit (1) and associated electronics are incorporated in the same unit, so that the heart rate is measured at a point.

13. A system according to claim 10, wherein the processor unit (2) is connected to at least a second sensor in the sensor unit (1) or at least a second sensor unit, such as an electroencephalographic sensor, an electromyographic sensor, an electrocardiographic sensor, a gyrometer or an accelerator, intended to measure at least another physiologically, neurologically or muscularly created signal, such as breathing, temperature, perspiration, muscular tensions, tremors/convulsions or galvanic skin response.

14. A system according to claim 13, wherein the sensor or sensor unit is connected to the analysis unit (5) optionally via at least a second verification unit.

15. A method of predicting epileptic seizures, comprising the following steps,
recording a physiologically, neurologically or muscularly created signal on the body of a user by means of a sensor unit (1),
comparing the sensor signal with reference parameters in a processor unit (2) and generating an indication signal when an alarm state is recorded, and
generating an alarm signal in an alarm unit (3),
the signal is measured continuously and compared with predetermined parameters characteristic of the measured signal, and only verified measurements are stored and processed in the processor unit (2), [p. 8, 1. 21-25, p. 11, 1. 8-19] and
that the processor unit (2) continuously compares the present measurement with one or more preceding measurements to change the value of the indication signal, which is transmitted to the alarm unit (3).

16. A method according to claim 15, wherein the processor unit (2) compares the verified measurement with the reference parameters and generates a first indication signal indicating whether a preictal phase is present or not.

17. A method according to claim 16, wherein the processor unit (2) determines the value of a second indication signal, which is transmitted further on to the alarm unit (3), on the basis of the present value of the first indication signal and the previous value or at least two of the preceding values of the first indication signal.

18. A method according to claim 17, wherein the value of the second indication signal is increased by a predetermined value, either if the present value and the previous value or at least two of the preceding values of the first indication signal are high, or if the number of high values is greater than the number of low values.

19. A method according to claim 17, wherein the value of the second indication signal is reduced by a predetermined value, either if the present value and the previous value or at least two of the preceding values of the first indication signal are low, or if the number of low values is greater than the number of high values.

20. A method according to claim 17, wherein the value of the second indication signal remains unchanged, either if the present value and the previous value of the first indication signal are different, or if the number of low values is equal to the number of high values.

21. A method according to claim 17, wherein the values of the first indication signal are summed, and the sum is compared with one or more threshold values indicating whether the value of the second indication signal is increased, is reduced or remains unchanged.

22. A method according to claim 15, wherein the signal is measured under various impacts normal conditions and is stored in a database as reference signals, and the reference parameters are updated automatically and possibly new reference parameters are added to the database by means of a self-learning process implemented in the processor unit (2) [p. 11, 1. 30-p. 12, 1. 10].

23. A method according to claim 15, wherein a heart rate sensor measures an electrocardiographic signal of the heart rate or detects another signal representative of the heart rate, such as pulse, blood pressure or a photoplethysmographic signal, or a first sensor measures another physiologically, neurologically or muscularly created signal than the heart rate, such as breathing, temperature, perspiration, muscular tensions, tremors/convulsions or galvanic skin response.

24. A method according to claim 23, wherein the heart rate is measured at a point by means of an electrode or a sensor in the sensor unit (1), which transmits data further on to the processor (2) either via a cable or wirelessly.

25. A method according to claim 23, wherein at least a second sensor in the sensor unit (1) or at least a second sensor unit measures at least another physiologically, neurologically or muscularly created signal, such as breathing, temperature, perspiration, muscular tensions, tremors/convulsions or galvanic skin response.

26. A method according to claim 25, wherein the measurement from the second sensor or sensor unit is compared with the measurement of the first signal in the analysis unit (5).
